(19) United States
(12) Patent Application Publication
(10) Pub. No.: US 2023/0128384 A1
(43) Pub. Date: Apr. 27, 2023

(54) DIALYSIS GARMENT

(71) Applicant: Mikel Hooks, Arcadia, CA (US)

(72) Inventor: Mikel Hooks, Arcadia, CA (US)

(21) Appl. No.: 17/508,975

(22) Filed: Oct. 23, 2021

(51) Int. Cl. A41D 13/12; A41D 27/10; A41D 27/28

(52) U.S. Cl. CPC A41D 13/1245; A41D 27/10; A41D 27/28; A61M 1/14

(57) ABSTRACT

A dialysis garment is provided. The garment includes opposed openings formed in the upper aim/shoulder area of the garment. The openings, which are selectively closeable, allow for the insertion of dialysis equipment while the garment is worn by the patient. The garment itself is insulated and may includes removable linings. The openings are configured to appear as aesthetic alterations.

### FIELD OF THE INVENTION

The present invention relates generally to medical garments. More particularly, the invention relates to a medical garment useful for dialysis patients.

### BACKGROUND OF THE INVENTION

Many patients presently receive hemodialysis, referred to herein as dialysis, for the treatment of kidney dysfunction and failure. A great number of the patients undergoing dialysis are elderly and suffer from poor blood circulation. Such elderly patients commonly must be covered with blankets to keep the patients warm and comfortable during dialysis since common coats and jackets must be removed during treatment to enable access to the arms of the user for connection of tubes used in dialysis.

A dialysis patient must wear a short sleeve shirt to provide access to the arms for connection of tubes used in dialysis. Accordingly, both arms of the patient are exposed for the entire duration of the treatment in what is typically a very cool room. The patient is instructed to stay warm by wearing clothes that can easily expose their access site, to cover with a blanket, or provide other means for staying warm. This situation can be very inconvenient especially for patients that are elderly or suffer from limited mobility.

Prior art attempts to deal with the problem suffer from a number of drawbacks. Typically, the warming garment is too open to provide adequate warmth for the user, especially the arm of the user receiving the IV needle. Also, many of the prior art garments are so radically altered they are not useful as normal apparel.

### SUMMARY OF THE INVENTION

A dialysis garment is provided. The garment includes opposed openings formed in the upper arm/shoulder area of the garment. The openings, which are selectively closeable, allow for the insertion of dialysis equipment while the garment is worn by the patient. The garment itself is insulated and may includes removable linings. The openings are configured to appear as aesthetic alterations.

it is a major object of the invention to provide an improved dialysis garment.

It is another object of the invention to provide an improved dialysis garment that can be worn during dialysis treatments.

It is another object of the invention to provide an improved dialysis garment that can be worn as an outer garment.

It is another object of the invention to provide an improved dialysis garment that has closeable access openings in an upper arm/shoulder area of the garment.

It is another object of the invention to provide an improved dialysis garment that has removable thermal linings,

### BRIEF DESCRIPTION OF THE DRAWINGS

FIG. 1 is a front view of the garment of the invention.

FIG. 2 is a front view of the garment of the invention with the access flaps open.

FIG. 3 is a front view of the garment of the invention showing a thermal liner.

### DETAILED DESCRIPTION

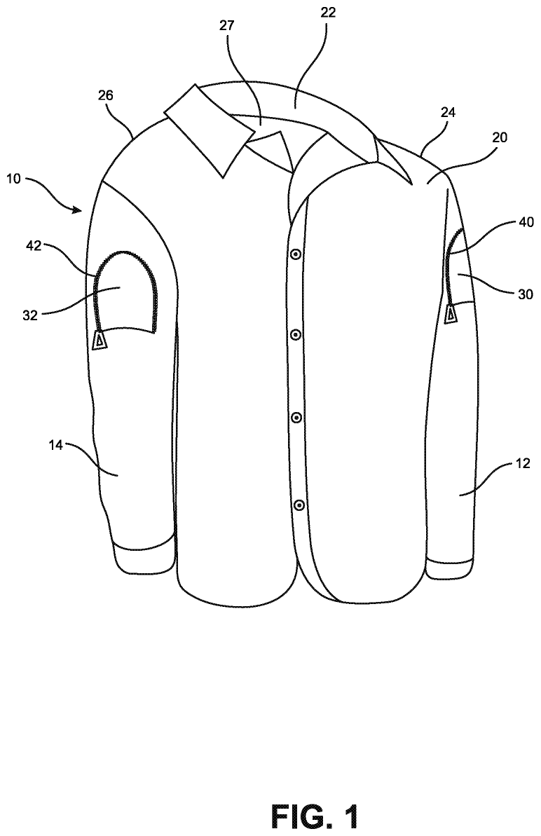

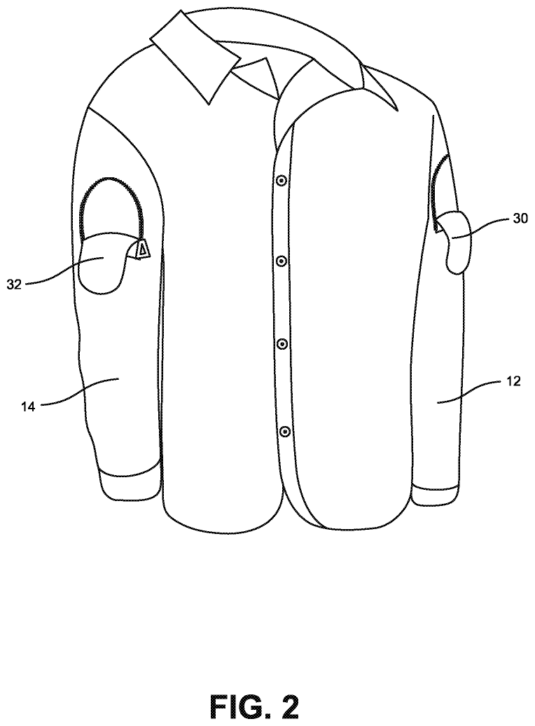

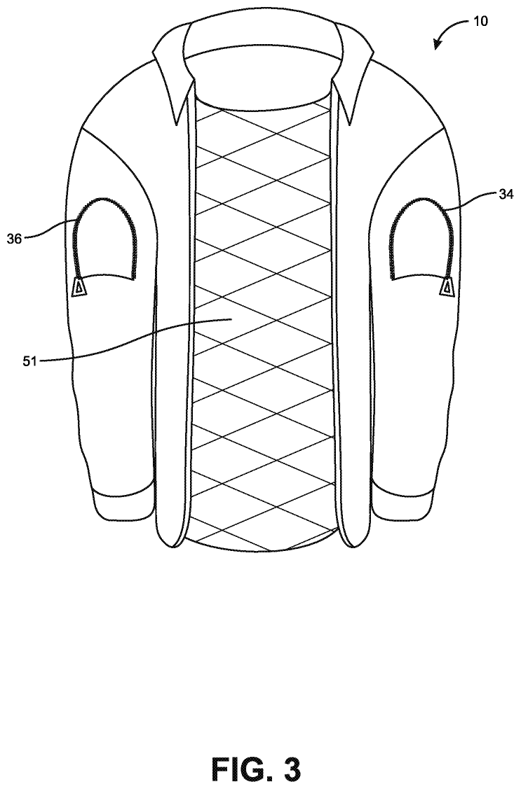

The present invention is directed to an outer garment that may be used as a thermal dialysis garment. Referring now to FIGS. 1-3, the garment, generally indicated by the numeral 10 can be a jacket, a sweater, a sweatshirt, or the like. The garment 10 extends at least to a waistline of a but can he extended further. Sizes can be standard (e.g., small, medium, large, and extra-large) or custom fitted. The garment 10 can be used as street. clothing as well, and can incorporate many aesthetic elements of fashion design. The garment can be made from any suitable (sanitzable) material for and also comfortable for extended use and temperatures. The garment 10 includes first and second sleeves 12,14, and a garment body 20. The garment body 20 has a neckline 22 between shoulders 24, 26 with a neck opening 10.

It can be seen that the garment 10, with its opposing shoulders 24, 26 has a corresponding pair of access flaps 30, 32. The flaps 30, 32 have zippered openings 34, 36 to allow for access to the patient's arm. The zippered openings 34, 36 have a U-shape roughly corresponding to the shape of the upper sleeve 12 of the garment. This shape provides a more aesthetic look while also functionally providing a large enough opening for the insertion of dialysis equipment. Prior art garments typically have elongated openings which are difficult to close and thus the thermal effectiveness of the garment is greatly reduced. Also, the access openings in prior art garments are positioned near the end of the sleeve making closure of the garment difficult when the dialysis equipement is inserted.

When in the open position, the access flaps 30, 32 hang down from the weight of the closure fastener 40, 42, which are preferably metal zippers but may optionally be buttons, clasps, or velcro. This hanging action allows for access to the patient's upper and lower arms without the constant hassle of having to reposition the material of the garment,

The garment 10 includes at least one thermal lining 50. Preferably, there are two linings 50, one for warm weather and another for colder weather. The linings 50 can be zipped out or otherwise removed to allow the user to replace when needed. Thus, an all season thermal dialysis garment is provided.

In use, the garment 10 can be worn into the dialysis room, The patient can then just unzip the flaps 30, 32 and allow the material to hang down to allow access to the patient's arm.

I claim:

1. A dialysis garment comprising:
a main body with a first sleeve, a second sleeve and a lower hem, each of the first and second sleeves including an access opening proximate a shoulder portion of said main body;
said access opening including a flap with a fastener;
whereby said flap hangs down when open to allow for access with medical equipment.
